(19) United States
(12) Patent Application Publication
(10) Pub. No.: US 2021/0137636 A1
(43) Pub. Date: May 13, 2021

(54) DEVICE FOR PROVIDING AN INTERFACE BETWEEN A MEDICAL INSTRUMENT AND A PART OF A HUMAN OR ANIMAL BODY AND METHODS OF USE THEREOF

(71) Applicant: UCL Business Ltd, London (GB)

(72) Inventors: Agostino STILLI, London (GB); Danail STOYANOV, London (GB)

(73) Assignee: UCL Business Ltd, London (GB)

(21) Appl. No.: 17/103,288

(22) Filed: Nov. 24, 2020

Related U.S. Application Data
(63) Continuation of application No. PCT/GB2019/051463, filed on May 29, 2019.

(30) Foreign Application Priority Data
May 29, 2018 (GB) 1808728.8

(51) Int. Cl. A61B 90/50; A61B 8/08; A61B 8/00; A61B 90/00; A61B 90/11

(52) U.S. Cl. CPC A61B 90/50; A61B 8/0833; A61B 90/11; A61B 90/08; A61B 8/4455

(57) ABSTRACT

A device for providing an interface between a medical instrument and a part of a human or animal body, the device comprising: at least one connecting part configured to allow attachment of the medical instrument thereto; and an attachment device configured to detachably attach the connecting part to the part of the human or animal body.

### CROSS REFERENCE TO RELATED APPLICATIONS

This continuation application claims priority benefit from International Application No. PCT/GB2019/051463 filed on May 29, 2019, which claimed priority from Great Britain Application No. 1808728.8 filed May 29, 2018, each of which is incorporated herein by reference in its entirety.

### FIELD OF THE INVENTION

The present invention relates to a device for providing an interface between a medical instrument and a part of a human or animal body. In particular, the invention relates to such devices for use during surgery on the human or animal. In some examples, the device may be used for guiding a medical instrument relative to a part of a human or animal body. In other examples, the device may be used for manipulating a part of a human or animal body.

### BACKGROUND OF THE INVENTION

Robotic-Assisted Partial Nephrectomy (RAPN) is a medical procedure in which part of a subject's kidney is removed. RAPN procedure include an identification phase of the tumour/mass to be removed and an outlining of the margins of resection using a surgical marker (e.g. an electrocautery tool). Intra-operative methods used for identification are generally limited to Ultrasound (US) imaging, which is cost-effective and safe for the subject. Pre-operative assessment for diagnosis and macro-localisation of the tumour is typically performed with Computer Tomography (CT) and/or Magnetic Resonance (MR) imaging. The use of drop-in US probes for RAPN procedures is widely recognized as the golden standard for the intra-operative detection and margins outlining of the tumour/mass targeted.

Typically, the probe is inserted through a standard trocar port (10 to 14 mm in diameter, depending on the brand of the selected probe) and the surgeon grasps it with a gripper, picking up the probe from a dedicated slot. Typically, the slot is designed to match the EndoWrist® Prograsp™ Forceps gripper of the daVinci Surgical System, (Intuitive Surgical Inc., Sunnyvale, Calif., US). Once the probe is connected with the gripper, the surgeon can navigate it to the target organ to perform the scan; after multiple swipes with the US probe on the kidney surface, the resection area is measured and marked with an electrocautery tool.

However, even with the dexterity that the robotic system provides the surgeon, the tumour localization and marking procedure can be challenging. Indeed, the US probe often requires repositioning because of slippage from the target organ surface, and more importantly, identification can be insufficient when the tumours are in challenging locations. In such cases, kidney repositioning could be required. Furthermore, a highly skilled surgeon is typically required to successfully perform this pre-operatory procedure. Similar problems exist in many other medical procedures.

It is an aim of the present invention to at least partially address some of the problems discussed above.

### SUMMARY OF THE INVENTION

According to a first aspect of the invention, there is provided a device for providing an interface between a medical instrument and a part of a human or animal body, the device comprising: at least one connecting part configured to allow attachment of the medical instrument thereto; and an attachment device configured to detachably attach the connecting part to the part of the human or animal body.

According to second aspect of the invention, there is provided a device wherein the at least one connecting part of the first aspect comprises a protrusion configured to allow attachment of the medical instrument thereto. Optionally, the attachment device is elongated and the protrusion extends substantially along the length of the attachment device. Optionally, the protrusion is configured to allow attachment of the medical instrument thereto at any point. Optionally, the protrusion is a ridge.

Optionally, the at least one connecting part comprises a guide path configured to support the medical instrument and constrain the movement of the medical instrument to a predefined range.

According to a third aspect of the invention, there is provided a device for guiding a medical instrument relative to a part of a human or animal body, the device comprising: a guide path configured to support the medical instrument and constrain the movement of the attached medical instrument to a predefined range; and an attachment device configured to detachably attach the guide path to the part of the human or animal body.

In any of the above aspects, optionally, the guide path is configured to allow attachment of the medical instrument thereto.

Optionally, the guide path comprises a rail. Optionally, the rail comprises at least one protrusion and/or at least one recess configured to engage with a corresponding part associated with the medical instrument. Optionally, the rail comprises two protrusions on two opposing sides of the rail. Optionally, the rail has a substantially T-shaped cross-section. Optionally, the rail has a uniform cross-sectional shape.

Optionally, the guide path is provided at a perimeter of the device.

Optionally, the attachment device is configured to be actuated between an attached state and a detached state. Optionally, the attachment device is pneumatically actuated.

Optionally, the attachment device comprises at least one suction cup.

Optionally, the device comprises a channel configured to be attached to a pressure source and the at least one suction cup is in communication with the channel.

Optionally, the device is formed from a flexible material.

Optionally, the device further comprises a connector attached to the guide path and configured to move along the guide path, the connector being integrated with or configured to attach to the medical instrument.

According to a fourth aspect of the invention, there is provided a connector for use with the device of the previous aspects, the connector comprising: a first attachment part configured to attach to the guide path of the device and allow the connector to move along the guide path.

Optionally, the connector further comprises a second attachment part configured to attach to the medical instrument. Alternatively, the connector is integrated with the medical instrument.

Optionally, the first attachment part is configured to engage with at least one protrusion and/or at least one recess of the guide path.

Optionally, the first attachment part comprises a roller configured to roll against a portion of the guide path as the connector moves along the guide path. Optionally, the roller is configured to be driven so as to move the connector along the guide path.

According to a fifth aspect of the invention there is provided a kit of parts comprising the device of the first, second or third aspects and the connector of the fourth aspect.

Optionally, the kit of parts further comprises a medical instrument.

In any of the above aspects, the medical instrument is optionally an ultrasound probe.

According to a sixth aspect of the present invention there is provided a method of manipulating a part of a human or animal body, the method comprising: attaching the device of any of the first to third aspects to the part of the human or animal body; connecting the medical instrument to the connecting part of the device; moving the medical instrument to manipulate the part of the human or animal body while the medical instrument is connected to the device.

According to a seventh aspect of the invention there is provided a method of guiding a medical instrument relative to a part of a human or animal body, the method comprising: attaching the device of the third aspect to the part of the human or animal body; supporting the medical instrument using the guide path of the device; and moving the medical instrument relative to the part of the human or animal body while the medical instrument is supported by the guide path.

In any of the above aspects, optionally, the medical instrument is an ultrasound probe.

Optionally, the part of the human or animal body is an internal surface.

### BRIEF DESCRIPTION OF THE DRAWINGS

The accompanying drawings, which are incorporated in and constitute a part of the specification, illustrate embodiments of the invention and together with the detailed description herein, serve to explain the principles of the invention. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the invention.

FIG. 1 is a perspective view of an example device;

FIG. 2 is a plan view of the example device;

FIG. 3 is a first cross section (transverse plane C-C) through the example device;

FIG. 4 is a first end view (proximal end) of the example device;

FIG. 5 is a second view (distal end) of the example device;

FIG. 6 is a second cross section (longitudinal plane A-A) of the example device;

FIG. 7 is a longitudinal cross section of the example device showing fluid flow; through the device;

FIG. 8 shows the example device attached to a first example medical instrument;

FIG. 9 is a perspective view of an example connector;

FIG. 10 is a cross section (longitudinal plane)

FIG. 11 is a perspective view of the example connector attached to an example medical instrument;

FIG. 12 shows the example device attached to a second example medical instrument;

FIG. 13 shows the example device attached to a third example medical instrument;

FIGS. 14A and 14B are a perspective views of a second example device;

FIGS. 15A and 15B are a perspective views of a third example device.

### DETAILED DESCRIPTION OF THE INVENTION

FIGS. 1 to 6 show various views of an example device 1 in accordance with the invention. The device 1 may be used for guiding a medical instrument 5 relative to a part of a human or animal body. The device 1 comprises a guide path 2 configured to support the medical instrument 5 (e.g. allow attachment of thereto) and constrain the movement of the attached medical instrument 5 to a predefined range.

The guide path 2 may provide a path along which the (e.g. attached) medical instrument 5 is able to move. The guide path 2 may allow movement of the attached medical instrument 5 in a direction tangential to the guide path 2 at a given location along the guide path 2. The guide path 2 may not allow movement, or allow limited movement, of the attached medical instrument 5 in a direction orthogonal to the guide path 2 at a given location along the guide path 2. The movement of the medical instrument 5 along the guide path 2 may be constrained by the support provided (e.g. the connection between) the medical instrument 5 and the guide path 2.

As shown in FIG. 1, the guide path 2 may be provided at a perimeter of the device 1. In other words, the guide path 2 may be provided along an edge of the device 1. However, the device 1 is not limited to such arrangements.

The guide path 2, may be provided in any shape. The shape may be selected according to the medical procedure for which the device 1 will be used. The shape may be selected based on the subject for which the device 1 will be used. In the example device 1 shown in FIG. 1, the guide path 2 is provided in a loop. The loop in this example comprises two straight portions joined by a curved portion, e.g. a U-shaped loop. However, alternatively, the guide path 2 may be looped, non-looped, straight or curved. In the example device 1 shown in FIG. 1, the guide path 2 is provided in an open loop. The open loop allows easy attachment/detachment of the medical instrument 5 at the ends of the guide path 2. This will be described further below. However, a closed loop may alternatively be used. In the example of FIG. 1, the guide path 2 is continuous. However, in other examples, the guide path 2 may be discontinuous, e.g. gaps may be provided in the guide path 2. The gaps may allow attachment/detachment of the medical instrument 5 to the guide path 2. Multiple devices may be configured to be arranged in series and may be attached to the same or different part of the human or animal body, in order to create a longer path.

The device 1 (e.g. specifically the guide path 2) may be formed from a flexible material, such that the shape of the guide path 2 changes as the device 1 flexes. Alternatively, the guide path 2 may be rigid, i.e. formed from a rigid material.

In the example device 1 shown in FIG. 1, the guide path 2 comprises a rail. In other words, a rail may function as the guide path 2. However, this is just one example and other examples of a guide path 2 may be used instead. The rail 2 may provide support for the medical instrument 5. The rail 2 may have a shape configured to engage with a corresponding part associated with the medical instrument 5 (e.g. a connector as described below). Accordingly, the rail 2 may comprise at least one protrusion and/or at least one recess.

For example, as shown in FIGS. 3 and 4, the rail 2 may comprise two protrusions 21 on two opposing sides of the rail 2. These two protrusions 21 may be aligned with each other, as shown in FIGS. 3 and 4, or may be offset with respect to each other. Adjacent to the protrusions may be respective recesses 22. Accordingly, as shown, the rail 2 may have a substantially T-shaped transverse cross-section (a plane perpendicular to the extension direction of the rail 2). Alternatively, any other suitable arrangement of protrusions and/or recesses may be used. The shape (e.g. cross-sectional shape) of the rail 2 is preferably substantially uniform along the length of the rail 2.

The device 1 further comprises an attachment device 3 configured to detachably attach the guide path 2 to a part of the human or animal body (subject). The attachment device 3 may be configured to attach the device 1 to a part of the subject such that the device 1 is fixed in position relative to the part of the subject. The attachment device 3 may be configured to selectively attach to and/or detach from the part of the subject. For example, the attachment device 3 may be configured to be actuated (e.g. pneumatically and/or mechanically) between an attached state and a detached state. Preferably, the attachment device 3 is configured so as not to cause damage or injury to the subject during attachment and/or detachment of the device 1.

As shown in FIG. 2, the attachment device 3 may comprise at least one suction cup 31. As shown in FIG. 3, each suction cup 31 may define a cavity. The cavity may be in pneumatic connection with a pressure source (not shown). The pressure source (e.g. a vacuum pump) may provide a negative pressure to the cavity (e.g. relative to the ambient pressure) such that the suction cup 31 attaches to a part of the subject. As shown in FIGS. 3 and 6, the device 1 may comprise a channel 32 configured to be attached to a pressure source. Each suction cup 31 may be in communication (e.g. pneumatic connection) with the channel 32, e.g. via sub-channels. In other example devices 1, multiple channels 32 may be provided. Each of these channels 32 may be connected to all, or some, of the suction cups 31. FIG. 7 shows a longitudinal cross section through the device 1, together with arrows showing a fluid flow (e.g. of air) through the device 1. It can be seen that the fluid flow is from the suction cups 31, through the channel 32 to the pressure source.

The suction cup 31 and cavity may be of any suitable shape. For example, the suction cups 31 may be substantially dome shaped, e.g. having a substantially circular cross-section. Alternatively, the suction cups 31 may be substantially pyramid shaped e.g. having a substantially polygonal (e.g. square, trapezium or triangular) cross-section.

In the example device 1 shown in the Figures, multiple suction cups 31 may be provided. These may be provided in a line, e.g. parallel to a longitudinal axis of the device 1. Accordingly, the channel 32 may be a substantially straight channel, e.g. extending in the longitudinal direction of the device 1. Further, as shown by FIGS. 3 and 5, a single line of suction cups 31 may be provided. However, any arrangement of suction cups 31 and corresponding shaped channels 32 may be used instead.

As shown in FIG. 3, for example, the device 1 may comprise a main body to which the guide path 2 and the attachment device 3 are connected. The main body may comprise a protrusion 11 configured to allow attachments of a gripper 51 thereto, for positioning the device 1.

The main body may comprise the channel 32, i.e. the main body may comprise material forming walls defining the channel 32. The channel 32 may be open at a proximal end of the channel 32, connectable to the pressure source, and closed at a distal end of the channel 32.

In some example devices, not shown in the Figures, the main body of the device 1 may comprise further channels and/or pressure chambers. These further channels and/or pressure chambers may be configured such that they can be selectively pressurised and depressurised in order to actively change the shape of the device 1. The shape of the device 1 may be actively changed in this manner after attachment to the part of the subject, in order to modify the shape of the part of the subject. In one example, pressure chambers on a first side of the device 1 may be pressurised, while pressure chambers on a second opposite side of the device 1 may be depressurised, such that the device 1 bends towards the second side.

The device 1 may be formed from a flexible material. This may allow the device 1 to conform to the shape of the part of the subject to which it is attached and/or modify the shape of the part of the subject. The flexible material may be a resilient material such as an elastomer, e.g. silicone or a rubber-like material. In particular, the suction cups 31 may be formed from a flexible material. However, other parts of the device 1 (e.g. the main body and/or guide path 2) may also be formed from a flexible material. The materials forming the suction cups 31 and other parts of the device 1 may be different.

For example, the suction cups 31 may be formed from a more flexible material than materials forming other parts of the device 1. Accordingly, the suction cups 31 may be formed from a material having a relatively low Shore hardness compared to materials forming other parts of the device 1. For example, the material forming the walls of the channel 32 may have a relatively high Shore hardness compared to the material forming the suction cups 31. This may ensure that the channel 32 does not collapse under low pressure. The material forming the guide path 2 may have a relatively high Shore hardness compared to the material forming the suction cups 31. This may ensure that the guide path 2 is able to support the medical instrument 5. The guide path 2 and the walls of the channel 32 may be formed from the same material and have the same Shore hardness. Shore harness can be measured using the standard test ASTM D2240.

The method of manufacturing the device 1 is not limited to a particular method. Manufacturing methods used may include injection moulding, casting, and additive manufacturing methods such as 3D printing. Multiple different types of materials may be co-moulded or 3D printed to form a single device, for example.

However, other materials may be used alternatively or additionally. For example, electro-active polymers may be used, the shape of which can be controlled electrically. Shape-memory alloys may be used. Shape memory polymers may be used. Low melting point alloys may be used. Magnetic materials may be used. The materials used may preferably be biocompatible materials.

FIG. 8 shows an example medical instrument 5 attached to an example device 1 in accordance with the invention. In the example shown, the medical instrument 5 is attached to the device 1 via a connector 4 configured to attach (e.g. detachably) to the medical instrument 5. The connector 4 is configured to be attached to the guide path 2 and configured to move along the guide path 2. As in the example shown, the connector 4 may be attached to the guide path 2 in a detachable manner (i.e. such that the connector 4 can be detached from the guide path 2). However, alternatively, the connector 4 may be attached to the guide path 2 in a fixed manner (i.e. such that the connector 4 cannot be detached from the guide path 2).

An example connector 4 is shown in further detail in FIGS. 9 to 11. The connector 4 comprises a first attachment part 41 configured to attach to the guide path 2 of the device 1 and allow the connector 4 to move along the guide path 2. The first attachment part 41 may have a shape configured to engage with the guide path 2 (e.g. the rail 2 described above). For example, the first attachment part 41 may be configured to engage with at least one protrusion and/or at least one recess of the guide path 2. Accordingly, the connector 4 may comprise recesses corresponding to protrusions of the guide path 2 and/or protrusions corresponding to recesses of the guide path 2. Adjacent surfaces of the first attachment part 41 and the rail 2 may slide against each other as the medical instrument 5 moves along the guide path 2.

As shown in the example connector 4 of FIGS. 9 to 11, the first attachment part 41 may comprise two opposing recesses 44 corresponding to the two opposing protrusions 21 of the rail 2. Adjacent the recesses 44 may be respective protrusions 45 corresponding to the recesses 22 of the rail 2. Accordingly, as shown, the first attachment part 41 may comprise a hollow space having a cross-sectional shape substantially corresponding to the cross-sectional shape of the rail 2, e.g. a substantially T-shaped transverse cross-section. As in the example shown in FIG. 6, the rail 2 may slot into the connector 4. The first attachment part 41 may slot onto and off the rail 2 at the ends of the rail 2. However, alternative mechanisms may be used to attach the first attachment part 41 to the rail 2. For example, the first attachment part 41 may be divided into two sections, each including one of the recesses 44 and respective protrusions 45. The sections may be configured to open (e.g. via a hinge, optionally spring-loaded) to allow the first attachment part 41 to move over the rail 2, then close to attach to the first attachment part 41 to the rail 2.

As shown in the example connector 4 of FIG. 11, the first attachment part 41 may further comprise a roller 43 configured to roll against a portion of the guide path 2 as the connector 4 moves along the guide path 2. The roller 43 may be configured to roll against an edge 23 of the rail 2. In the example shown in FIG. 1, the roller 43 may roll against the portion (e.g. edge 23) of the rail 2 forming the top surface of the T-shaped cross-section. In an example connector not shown, the roller 43 may be driven so as to move the connector 4 along the guide path 2. The roller 43 and portion of the guide path 2 may be configured to engage with each other, e.g. via respective corresponding protrusions/recesses or teeth forming a rack and pinion arrangement.

The connector 4 shown in FIGS. 9 to 11 further comprises a second attachment part 42 configured to attach to the medical instrument 5. The specific configuration of the second attachment part 42 depends on the type of medical instrument 5. For example, the second attachment part 42 of the connector 4 shown in FIGS. 9 to 11 comprises a recess 46 configured to accommodate part of the medical instrument 5. The attachment part 42 may further comprise a slot 47 configured to engage with a protrusion 53 of the medical instrument (e.g. usually for attachment of a gripper 51 thereto). The slot 47 may be provided in a wedge shaped portion 48 of the connector 4. The protrusion 53 of the medical instrument 5 may comprise a flange at a proximal end of the protrusion 53. The wedge shaped portion 48 may be configured to slot over the protrusion 53, beneath the flange. The engagement between the slot 47 and the protrusion 53 may provide a secure connection. Alternatively, or additionally, the recess 46 may be configured to have a friction fit with the medical instrument 5. However, other arrangements may be used instead, e.g. a snap fit arrangement. The connector 4 may be configured to allow direct attachment of a gripper 51 thereto, e.g. via a protrusion 49 or ridge on the connector 4.

FIGS. 12 and 15 show further examples of medical instruments 5 attached to an example device 1 in accordance with the invention. In the examples shown, the medical instrument 5 is attached to the device 1 via a connector integrated with the medical instrument 5. In these examples, the medical instrument 5 comprises an attachment part 52 configured to attach to the guide path 2 of the device 1 and allow the medical instrument 5 to move along the guide path 2. The attachment part 52 may be the same as the first attachment part 41 of the connector 4 described above. The medical instrument 5 comprises a protrusion 53 for attachment of a gripper 51 thereto.

FIG. 12 shows an example medical device requiring a gripper 51 to move the medical instrument 5. On the other hand, FIG. 13 shows an example medical device 5 not requiring a gripper 51 to move the medical device 5, e.g. the medical device 5 may be moved directly, by e.g. a robotics system such as the daVinci Robot.

In another alternative example device, not shown in the Figures, the guide path 2 may comprise a magnetic material configured to allow magnetic attachment of the medical instrument 5 thereto. The magnetic guide path 2 may comprise a strip of magnetic material. The magnetic material may be ferromagnetic material or a non-ferromagnetic material. Otherwise, the device 1 may be the same as described above. The first attachment part 41 of the connector 4 or the attachment part 52 of the medical instrument 5 may accordingly comprise a magnetic material configured to magnetically attach to the guide path 2. Otherwise, the connector 4 and medical instrument 5 may be the same as described above.

In yet another alternative example device, the guide path 2 may be formed by a channel, within the device 1. The channel may be the channel 32 described above or a different channel. The medical instrument 5 may enter the channel via an opening and be supported and guided by the channel. The medical instrument may be flexible to allow it to bend, e.g. as the channel bends. Otherwise, the device 1 may be the same as described above.

The device 1 may be used to guide a medical instrument 5 relative to a part of a human or animal body by attaching the device 1 to the part of the human or animal body, attaching the medical instrument 5 to the guide path 2 of the device 1 (these previous steps may be performed in any order), and moving the medical instrument 5 relative to the part of the human or animal body, while the medical instrument 5 is attached to the guide path 2.

The medical instrument 5 is not limited to any particular type of medical instrument, provided that the medical instrument 5 can be connected to the guide path 2 (either directly or indirectly). The type of medical instruments may non-exhaustively include imaging probes (such as an ultrasound probe, a gamma-imaging probe or a beta-imaging probe), sensors, or surgical tools (such as an electrocautery tool or a cutting tool).

The part of the human or animal body is not limited to any particular type of body part. For example, the body part may non-exhaustively include the surfaces of internal organs (such as a kidney, liver, stomach, prostate, heart or spleen), or the surfaces of internal cavities (such as the abdominal, pelvic or thoracic cavities). The device 1 may be used during minimally invasive surgery, such as laparoscopic surgery.

The device 1 may have a substantially elongate shape, like the example device 1 shown in the Figures. This may be advantageous because the device 1 can be easily inserted into the body of the subject via a laparoscopic port. Standard laparoscopic parts have a diameter of around 10 mm to 14 mm. Accordingly, the dimensions of the device 1 may be configured to fit through such a port. For example, the width and depth of the device 1 may be such that the circumcircle of a transverse cross-section through the device 1 has a diameter less than 14 mm or less than 10 mm.

The device 1 may also be used to manoeuvre a body part to which it is attached. For example, an example device 1 is described above that can actively change the shape of the body part to which it is attached. Further, the device 1 may be used to move a body part to which it is attached, i.e. by directly moving the device 1 while attached.

FIGS. 14A and 14B show a second example device 101 in accordance with the present invention. The device 101 may be used for manipulating a part of a human or animal body, for example. The device 101 comprises an attachment device 103 and main body, which may be substantially the same as the attachment device 3 and main body described above. The device 101 further comprises a connecting part 111 configured to allow attachment of a medical instrument, such as a gripper 51, thereto. In this example, there is no guide path 2.

In this example device 101, the connecting part 111 comprises a protrusion e.g. from the main body of the device 101. The protrusion allows attachment of the medical instrument thereto. For example, the protrusion may allow a gripper 51 to grip the device 101.

As shown in FIGS. 14A and 14B, the attachment part 103 is elongate in shape. Therefore, as shown, the protrusion 111 may also be elongate in shape. In particular, the protrusion 111 may extend substantially along the length of the attachment device 103.

The protrusion 111, may be provided in any shape. The shape may be selected according to the medical procedure for which the device 1 will be used. The shape may be selected based on the subject for which the device 1 will be used. In the example device 101 shown in FIGS. 14A and 14B, the protrusion 111 is provided in a straight line. However, this may be curved instead.

The connecting part 111 may be provided substantially in a central portion of the main body of the device e.g. rather than being provided around the perimeter of the device like the guide path 2 described above. As shown the connecting part 111 may be a substantially central ridge.

The protrusion 111 may be configured to allow attachment of the medical instrument at any point. Accordingly, the protrusion 111 may have a substantially uniform transverse cross sectional shape along its length. The cross section of the protrusion may have a shape to aid gripping. For example, the cross-sectional shape may be tapered towards the distal end, as shown in FIG. 14A. Alternatively, the cross-sectional shape may be T-shaped, like the guide rails 2 described above.

The example device 101, shown in FIGS. 14A and 14B, shows an alternative arrangement to that shown in the previous Figures. In particular, rather than multiple suction cups 31, as shown in FIG. 2, for example, only one suction cup 131 is provided.

The suction cup 131 is provided substantially over the entirety of one side of the device 101, e.g. the main body. As shown, the suction cup 131 may surround multiple sub-channels 133 leading off a main channel (not shown) through the device 101 (the main channel corresponding to the channel 32 in the previous Figures). The suction cup 131 may be a skirt around the edge of the device main body. In other examples, multiple suction cups 131 may be provided each surrounding multiple sub-channels 133. In this example, the suction cup 131 has a substantially regular-trapezium transverse cross-sectional shape.

FIGS. 15A and 15B show a third example device 201 in accordance with the invention. In this example, the device 201 is a combination of the first example device 1 and second example device 101. In particular, as shown, the third example device 201 combines the connecting part (i.e. guide rail 2) of the first example 1 with connecting part 111 of the second example device 101. The example device 201 comprises attachment device 203 (with cup 231 and sub-channels 233) and connecting part 211, as in the second example device 101.

The guide rail 202 of the third example device may be substantially the same as the guide rail 2 of the first example device 1. In the example shown in FIGS. 15A and 15B, the guide rail 202 comprises a single protrusion 221, so as to form an L-shaped cross section, rather than two protrusion 21 forming a T-shaped cross section. It should be noted that this arrangement may alternatively be used in the first example device 1. In the Figures, the protrusion 221 points away from the attachment device 203 (or the part of the body, in use), i.e. upward. However, the opposite arrangement may be used, namely pointing towards the attachment device 203 (or the part of the body, in use), i.e. downward.

It should be understood that, unless otherwise stated, the features of the second and third example device 101, 201 are substantially the same as the features of the first example device 1. Accordingly, any features of the first example device 1, not specifically mentioned with respect to the second and third example devices 101, 201 should be understood to apply also to the second and third example devices 101, 201.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

It should be understood that variations of the above examples, including combinations of different examples are possible without departing from the scope of the invention.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

Having thus described the preferred embodiments, the invention is now claimed to be:

1. A device for providing an interface between a medical instrument and a part of a human or animal body, the device comprising:
at least one connecting part configured to allow attachment of the medical instrument thereto; and
an attachment device configured to detachably attach the connecting part to the part of the human or animal body.

2. The device of claim 1, wherein the at least one connecting part comprises a protrusion configured to allow attachment of the medical instrument thereto, optionally wherein the protrusion is a ridge.

3. The device of claim 2, wherein the attachment device is elongate, and the protrusion extends substantially along the length of the attachment device.

4. The device of claim 3, wherein the protrusion is configured to allow attachment of the medical instrument thereto at any point.

5. The device of claim 1, wherein the at least one connecting part comprises a guide path configured to support the medical instrument and constrain the movement of the medical instrument to a predefined range.

6. The device of claim 5, wherein the guide path is configured to allow attachment of the medical instrument thereto.

7. The device of claim 5, wherein the guide path comprises a rail, optionally wherein the rail comprises at least one protrusion and/or at least one recess configured to engage with a corresponding part associated with the medical instrument, optionally wherein the rail comprises two protrusions on two opposing sides of the rail.

8. The device of claim 7, wherein the rail has a substantially T-shaped cross-section.

9. The device of claim 7, wherein the rail has a uniform cross-sectional shape.

10. The device of claim 5, wherein the guide path is provided at a perimeter of the device.

11. The device of claim 1, wherein the attachment device is configured to be actuated between an attached state and a detached state, optionally wherein the attachment device is pneumatically actuated.

12. The device of claim 1, wherein the attachment device comprises at least one suction cup, optionally wherein the device comprises a channel configured to be attached to a pressure source and the at least one suction cup is in communication with the channel.

13. The device of claim 1, wherein the device is flexible.

14. The device of claim 5, further comprising a connector attached to the guide path, wherein the connector is configured to move along the guide path, the connector being integrated with the medical instrument or configured to attach to the medical instrument.

15. The device of claim 1, wherein the medical instrument is an ultrasound probe.

16. The device of claim 5, further comprising a connector, the connector comprising:
a first attachment part configured to attach to the guide path of the device and allow the connector to move along the guide path, optionally wherein the first attachment part is configured to engage with at least one protrusion and/or at least one recess of the guide path.

17. The device of claim 16, the connector further comprising a second attachment part configured to attach to the medical instrument.

18. The device of claim 16, wherein the connector is integrated with the medical instrument.

19. The device of claim 16, wherein the first attachment part comprises a roller configured to roll against a portion of the guide path as the connector moves along the guide path.

20. The device of claim 19, wherein the roller is configured to be driven so as to move the connector along the guide path.
